(19) United States
(12) Patent Application Publication
(10) Pub. No.: US 2026/0092879 A1
(43) Pub. Date: Apr. 2, 2026

(54) METHOD FOR DETERMINING ELECTROMAGNETIC PROPERTIES OF A NON-UNIFORM MEDIUM BY MEANS OF A RADIO-FREQUENCY DETECTION SYSTEM

(71) Applicant: COMMISSARIAT À L'ENERGIE ATOMIQUE ET AUX ENERGIES ALTERNATIVES, PARIS (FR)

(72) Inventors: Pascal PAGANI, GRENOBLE (FR); Jean-Baptiste DORE, GRENOBLE (FR); Raffaele D'ERRICO, GRENOBLE (FR)

(21) Appl. No.: 19/344,354

(22) Filed: Sep. 29, 2025

(30) Foreign Application Priority Data
Oct. 2, 2024 (FR) 2410590

(51) Int. Cl. G01N 22/00; G01N 33/24; G01S 7/41; G01S 13/42; G01V 3/12

(52) U.S. Cl. CPC G01N 22/00; G01N 33/24; G01S 7/418; G01S 13/42; G01V 3/12

(57) ABSTRACT

A method for determining electromagnetic properties of a non-uniform medium by a radio-frequency detection system is provided. The method for determining electromagnetic properties of a medium comprises the steps of: determining a measurement of a frequency transfer function of a transmission channel characterizing the medium; determining a model of the frequency transfer function; determining an estimation function estimating a correlation coefficient between the at least one measurement of a frequency transfer function and the model of the function; setting an initial value of the at least one electromagnetic property; searching for a set of first local maxima of the estimation function in the domain defined by the at least one positional variable for the initial value of the at least one electromagnetic property; for each first local maximum, searching for a second local maximum of the estimation function in the domain defined by the at least one variable characterizing the at least one electromagnetic property of the medium and in a domain defined by the at least one positional variable constrained by the first local maximum.

### CROSS-REFERENCE TO RELATED APPLICATION

This application claims priority to foreign French patent application No. FR 2410590, filed on Oct. 2, 2024, the disclosure of which is incorporated by reference in its entirety.

### FIELD OF THE INVENTION

The invention relates to the field of non-destructive devices and methods for characterizing the composition of a material or non-uniform medium by means of a radio-frequency system. For example, the invention in particular relates to ground-penetrating radars allowing underground targets to be detected, located or identified. The invention may also relate to the field of health, characterization of biological tissues for example.

More precisely, the invention relates to a method for determining electromagnetic properties of a non-uniform medium by means of a radio-frequency system. The proposed method allows a satisfactory resolution to be obtained with a decreased complexity compatible with implementation by a device with limited resources.

### BACKGROUND

In any application aiming to detect elements in a medium or material using a radio-frequency detection system, knowledge of the electromagnetic properties of the detection medium is important.

Specifically, the spatial location of sought targets is generally determined by analysing the propagation time of electromagnetic signals transmitted and received by the radio-frequency system, which are then converted into distances using an estimate of the propagation velocity of the electromagnetic waves through the medium. However, the propagation velocity of an electromagnetic wave depends on the electromagnetic properties of the medium, and in particular (although not exclusively) on its dielectric permittivity. The electromagnetic properties of the medium make it possible to describe the response of this medium to an applied electric field.

For example, in the case of ground-penetrating radars, the medium through which the electromagnetic waves pass is generally the ground. In the case of applications in the health field, the medium passed through is a set of biological tissues.

Most of the time, assumptions are made regarding the actual values of the electromagnetic characteristics of the medium, such as its dielectric permittivity. However, the accuracy of these values may be important to obtaining sufficient accuracy in the detection and characterization of targets.

The invention therefore addresses a general problem of accurate characterization of the electromagnetic properties of a medium through which an electromagnetic wave propagates, in particular its dielectric permittivity or permeability.

Moreover, another general problem in this context consists in carrying out this characterization with limited processing complexity so as to allow the method to be integrated into an embedded device.

In the field of ground-penetrating radars, there are a number of methods for detecting the position of a target.

One known prior-art method involves fitting hyperbolic curves, as for example described in reference [1]. When an antenna of the ground-penetrating radar is moved over the surface of the ground and the penetrating wave encounters buried targets or interfaces between various layers of the ground, the received signal observed in the time domain has a hyperbola shape, because of the propagation times of the reflected waves, which differ depending on the relative position of the target and the antennas of the radar. A number of configurations in respect of antenna positions may be used to observe this effect. For example, the transmit (Tx) and receive (Rx) antennas may make an identical movement, each remaining in a fixed position relative to the other. Alternatively, the Tx antenna may make a movement symmetrical to the movement of the Rx antenna, with respect to a fixed central point, this configuration then being called the “Common Mid Point” configuration. Lastly, one antenna may be placed in various positions with respect to a fixed second antenna, in a configuration called the “Wide Angle Reflection and Refraction” configuration.

All these observation techniques make it possible to obtain a hyperbola-shaped plot when observing the received signal in the time domain as a function of an antenna position metric. The peak of the hyperbola then indicates the actual position of the target (or interface) and the shape of the hyperbola depends on the horizontal spatial increment and on the velocity of the signal in the material: the higher the velocity, the wider the hyperbola and vice versa. By analysing the shape of the hyperbola, it is possible to determine the propagation velocity of the wave in the medium and thus determine the electrical permittivity of the medium. The observed signal is fitted to a theoretical hyperbola through semblance analysis, as described for example in references [2, 3].

This method is very commonly used to calibrate GPR data, but has a number of major drawbacks. The accuracy of the method remains low due to the difficulty of fitting a theoretical curve to an experimental observation marred by uncertainty. To increase accuracy a higher number of observations must be made, this potentially requiring the antennas to be moved for each observation. Antenna movement may be avoided if multiple, spatially distributed Tx and Rx antennas are available, but in this case the accuracy of the fitting technique is low. The method is also not well suited to detection of stratified layers in a non-uniform medium, because the various reflections produce hyperbolas that overlap, making fitting more difficult. This situation requires complex algorithms to be implemented, as illustrated in reference [4].

Another type of known method relates to migration methods, as for example described in reference [5]. These methods seek to reduce the inaccuracy in the hyperbola-fitting method due to observational uncertainties in the received signal. It is a form of mathematical processing, the main aim of which is to increase the accuracy of hyperbolic plots of targets and interfaces. Theoretically, this process makes it possible to reduce the hyperbolas acquired by the radar to a single location point. However, the implementation of this technique is difficult when applied to potentially noisy experimental data. There are a wide variety of different migration methods including hyperbolic summation, Kirchhoff's migration, back-projection focusing, phase shift migration, and ω-k migration [5].

The main parameter required to reduce a hyperbola to a point corresponds to the dielectric properties of the ground, as taught in reference [6]. This makes the migration process a way of increasing the accuracy of the fit to the curve since the process only works properly if the applied transmission velocity through the ground is accurate. Thus, migration methods use an average estimate of the relative permittivity of a material for a given depth. One drawback of these methods is their relative inaccuracy when the material is made up of a plurality of layers of different permittivities.

Reference [6]proposes a method applied to the detection of buried targets. It proposes to test different values of average permittivities for a given acquisition. The selected permittivity is the one that minimizes the area of the detected target. Therefore, this method is not generic and is not applicable in the absence of a target, for example in the case of a stratified non-uniform material. Moreover, the metric of the apparent area of the target is inexact and is suitable only for targets of simple shape, the apparent area of which varies monotonically with the absolute error in the assumed permittivity.

Other known methods aim to solve the problem of determining the electromagnetic properties of a medium using a radio-frequency detection system. Mention may in particular be made of the following patents and patent applications: WO2020180191, EP3164672, WO2022091456, FR3041108 and FR3142553.

All these methods have drawbacks; some are invasive and destructive, others require prior knowledge of the geometry of the medium, and yet others are not applicable to a non-uniform medium. Generally, these methods provide ways of processing radar data in the time domain.

There is a need for a method for accurately characterizing the electromagnetic properties of a medium, by means of a radio-frequency detection system, that overcomes the limitations of prior-art methods. In particular, the proposed method must be of low complexity in order to be compatible with implementation in an embedded system with limited resources.

A new characterizing method is provided that operates in the frequency domain and that has a lower complexity than the prior-art methods.

### SUMMARY OF THE INVENTION

To this end, one subject of the invention is a method for determining electromagnetic properties of a medium, comprising the steps of:
determining, by means of a radio-frequency detection system comprising at least one transmitter and one receiver, for each pair associating one receiver and one transmitter, a measurement of a frequency transfer function of a transmission channel characterizing said medium,
determining a model of said frequency transfer function dependent on at least one positional variable and on at least one variable characterizing the at least one electromagnetic property of the medium,
determining an estimation function estimating a correlation coefficient between the at least one measurement of a frequency transfer function and the model of said function,
setting an initial value of the at least one electromagnetic property,
searching for a set of first local maxima of the estimation function in the domain defined by the at least one positional variable for the initial value of the at least one electromagnetic property,
for each first local maximum, searching for a second local maximum of the estimation function in the domain defined by the at least one variable characterizing the at least one electromagnetic property of the medium and in a domain defined by the at least one positional variable constrained by the first local maximum.

According to one particular aspect of the invention, the search for a second local maximum of the estimation function is carried out over an interval of the at least one positional variable in the vicinity of the first local maximum.

According to one particular aspect of the invention, the search for a second local maximum of the estimation function is carried out in a constrained domain such that the electrical length, defined by the product of the square root of the real part of the dielectric permittivity and the total distance travelled by the signal between a transmitter and a receiver, remains substantially constant between the first local maxima and the second local maximum.

According to one particular aspect of the invention, the constraint of substantially constant electrical length is imposed for every pair associating one transmitter and one receiver.

According to one particular aspect of the invention, the constraint of substantially constant electrical length is imposed only for the pair associating one transmitter and one receiver that minimizes the distance travelled by a radio-frequency signal with a scatterer of coordinates equal to the first local maximum.

In one variant of embodiment, the method according to the invention further comprises a step of determining an image of the medium as a function of the at least one positional variable from said measurements and the at least one initial value, the search for said set of first local maxima being carried out on the image and not the estimation function.

According to one particular aspect of the invention, the steps of determining at least one measurement of the frequency transfer function and of determining a model of said transfer function are carried out for each pair associating one transmitter and one receiver of the detection system, the method further comprising determining a correlation coefficient between said measurement and said model for each of said pairs, the estimation function being calculated from all the correlation coefficients.

According to one particular aspect of the invention, the amplitude and phase of the model of the frequency transfer function depend on the distance between a transmit antenna and a point on the plane and the distance between the point on the plane and a receive antenna.

According to one particular aspect of the invention, the at least one variable characterizing an electromagnetic property of the medium is selected from: the real part or imaginary part of the dielectric permittivity or permeability.

According to one particular aspect of the invention, the correlation coefficient is determined using a ZF, MMSE or MRC equalization method.

Another subject of the invention is a device for determining electromagnetic properties of a medium comprising a radio-frequency detection system comprising at least one transmitter and one receiver and a processing unit, the system being configured to implement the method according to the invention.

According to one particular aspect of the invention, the radio-frequency detection system is a ground-penetrating radar.

Other subjects of the invention are a computer program comprising code instructions that cause the device according to the invention to execute the steps of the method according to the invention and a computer-readable medium on which the computer program according to the invention is stored.

### BRIEF DESCRIPTION OF THE DRAWINGS

Other features and advantages of the present invention will become more clearly apparent on reading the following description, with reference to the following appended drawings.

FIG. 1 shows a flowchart detailing the general principle of a method for determining electromagnetic properties of a medium according to the invention,

FIG. 2 schematically shows one example of a radio-frequency detection system capable of implementing the invention,

FIG. 3 illustrates a graphical representation of a transfer function model,

FIG. 4 shows a flowchart describing the steps of a method of optimization under constraints according to a first embodiment of the invention,

FIG. 4a shows a flowchart describing the steps of a method of optimization under constraints according to a second embodiment of the invention,

FIG. 5 shows a radar image of a region of ground in the plane (x,z),

FIG. 6 schematically shows one example of a system capable of implementing the invention.

### DETAILED DESCRIPTION

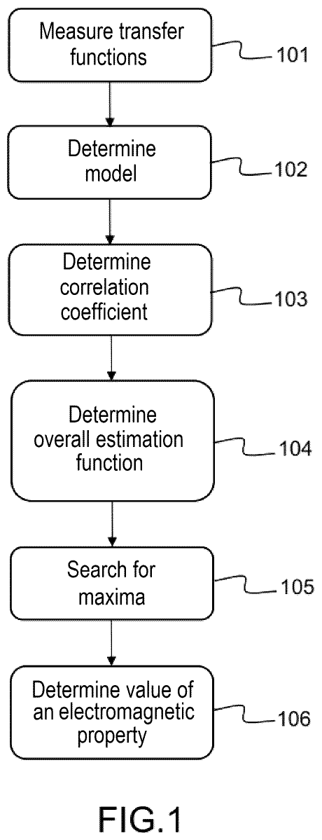

FIG. 1 shows a flowchart describing the general principle of a method for determining electromagnetic properties of a medium according to the invention.

In the remainder of the description, the method will be described in the context of a non-limiting example intended to determine the permittivity of a medium such as the ground by means of a radio-frequency detection system such as a ground-penetrating radar. However, the invention is not limited to determination of permittivity but extends to determination of any electromagnetic property characterizing a medium, such as also permeability.

Likewise, the invention is not limited to the use of a ground-penetrating radar but extends to any radio-frequency detection device.

The method starts in step 101 with a set of measurements of frequency transfer functions by means of a radio-frequency detection device.

The radio-frequency detection system is made up of a transmitter capable of transmitting a radio-frequency signal via at least one Tx antenna and of a receiver capable of receiving this radio-frequency signal via at least one Rx antenna. A number of versions of the transmitted signal are observed and depend on the positions of the Tx antenna and the Rx antenna.

According to one variant of embodiment, these various versions of the received signal are obtained from a plurality of Tx antennas located at fixed positions and a plurality of Rx antennas located at fixed positions, in a configuration of the radio-frequency detection device called the MIMO configuration, MIMO standing for “Multiple Input Multiple Output”.

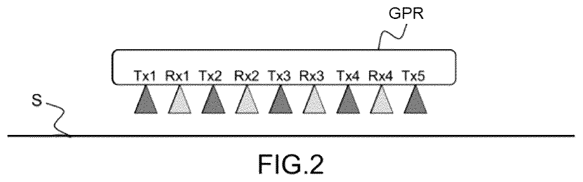

FIG. 2 schematically shows one example of such a radio-frequency detection system taking the form of a ground-penetrating radar (GPR) having five transmit and four receive antennas.

Alternatively, the various measurements may also be obtained by carrying out successive transmissions, and by moving the Tx or Rx antennas between each transmission.

Thus, one measurement is obtained for each pair associating one Tx antenna with one Rx antenna for a given position of these two antennas.

The invention is also applicable to the case where a single antenna serves both for transmission and reception and is connected to the transmitter and receiver by way of a separating device such as a coupler.

The signals transmitted via each pair of Tx and Rx antennas are separated in the time domain through successive transmissions, but they may also be separated using any conventional multiple-access method, such as frequency- or code-based separation.

The general case of a system made up of M transmit antennas and N receive antennas or more generally M different transmit antenna positions and N different receive antenna positions will be considered. M and N are two strictly positive integers.

The radio-frequency detection system further comprises an estimation module configured to determine, from the signal transmitted via the Tx antenna in position m and received via the Rx antenna in position n, a complex transfer function Hₘₙ(f) for various frequency values f.

A number of methods for obtaining the transfer function Hₘₙ(f) and for choosing a signal to be transmitted exist in the prior art and are techniques known in the field of channel estimation or radio sounding. References [8], [9] and [10] give examples of such methods.

The transfer function Hₘₙ(f) depends on the composition of the medium through which the signal passes, and in particular on the spatial distribution of the electromagnetic properties of the medium, and in the case of the presence of targets, on the position and radar cross section (RCS) of the targets.

At the end of step 101, one measurement of the transfer function Hₘₙ(f) is therefore obtained for each of a plurality of frequencies and for each pair (m,n) associating one transmit antenna and one receive antenna for which a measurement was taken.

In step 102, a theoretical model of the frequency transfer function that takes into account the electromagnetic properties of the medium through which the signal passes is then determined.

For example, the chosen model is suitable for a point scatterer located at a position rₚ in a non-uniform medium with a permittivity of real part ε′ᵣ and of negligible imaginary part. The transfer function between emitter m and receiver n is modelled by the following equation, Equation (1):

$${W}_{m n} ( f ) = \frac{\sqrt{{G}_{m} {G}_{n}} \cdot c}{f \cdot ‖ {r}_{p} - {r}_{m} ‖ \cdot ‖ {r}_{n} - {r}_{p} ‖ \cdot {( 4 π )}^{3 / 2} \cdot \sqrt{{ε′}_{r}}} \cdot {e}^{- i 2 π f \frac{\sqrt{{ε}_{r}^{′}}}{c} ( ‖ {r}_{p} - {r}_{m} ‖ + ‖ {r}_{n} - {r}_{p} ‖ )} \qquad ( 1 )$$

where rₘ and rₙ are the positions of the transmit antenna and receive antenna, respectively;
Gₘ and Gₙ are the respective gains of the transmit and receive antennas;
c is the speed of light in vacuum.

The model given by Equation (1) considers on the one hand the phase shift related to the geometry of the scene in the case of rectilinear propagation, and on the other hand an amplitude given by a radar equation taken from the literature (see reference [7]).

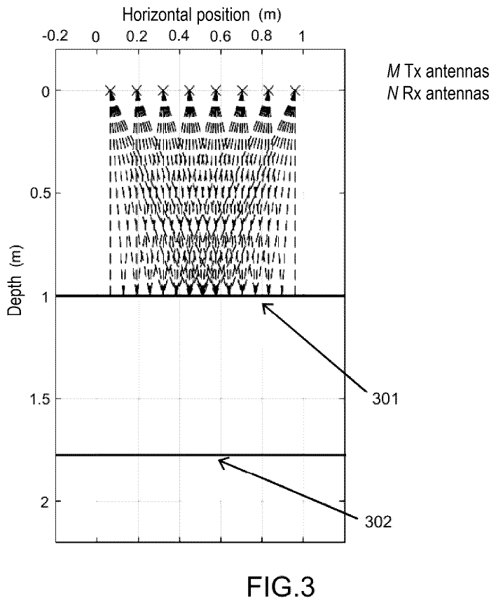

FIG. 3 schematically shows a graphical representation of the model of Equation (1) for M=N=8 and for two scatterers 301, 302. Reference 303 designates a test point located at the coordinates (x,z).

Without departing from the scope of the invention, other models may be developed to replace the one of Equation (1) provided that they take into account at least one electromagnetic property of the medium, for example the permittivity ε′ᵣ.

For example, Equation (1) may be replaced by the following Equation (1a) in which the coefficient \sqrt{ε′ᵣ} is replaced by ε′ᵣ

$${W}_{m n} ( f ) = \frac{\sqrt{{G}_{m} {G}_{n}} \cdot c}{f \cdot ‖ {r}_{p} - {r}_{m} ‖ \cdot ‖ {r}_{n} - {r}_{p} ‖ \cdot {( 4 π )}^{3 / 2} \cdot \sqrt{{ε′}_{r}}} \cdot {e}^{- i 2 π f \frac{\sqrt{{ε}_{r}^{′}}}{c} ( ‖ {r}_{p} - {r}_{m} ‖ + ‖ {r}_{n} - {r}_{p} ‖ )} \qquad ( 1 a )$$

Specifically, Equation (1) provides a transfer function model suitable for a point scatterer located at a position rₚ in a uniform medium the permittivity of which has a real part ε′ᵣ and a negligible imaginary part. Its phase is calculated by considering the delay accumulated by the wave in the case of rectilinear propagation between the Tx antenna and the scatterer, then between the scatterer and the Rx antenna. Its amplitude is given by the radar equation.

Observation of experimental results seems to show that the model given by Equation (1) does not always model amplitude optimally.

A physical explanation in respect of the modification proposed in Equation (1a) is that radar cross section (RCS) is assumed to be independent of the electromagnetic properties of the medium. However, it is believed that the RCS of a given object varies in a manner inversely proportional to the permittivity ε′ᵣ of the medium.

The model of Equation (1a) allows better focusing, as explained below.

In step 103, a correlation coefficient between the measurement made in step 101 and the model determined in step 102 is determined, for each pair of antennas.

For example, the correlation coefficient is determined by means of relationship (2) which represents ZF equalization, ZF standing for Zero Forcing.

$${ℒ}_{m n} ( f , {r}_{p} , {ε}_{r}^{′} ) = \frac{{H}_{m n} ( f ) \cdot {W}_{m n}^{*} ( f , {r}_{p} , {ε}_{r}^{′} )}{{\vert {W}_{m n} ( f , {r}_{p} , {ε}_{r}^{′} ) \vert}^{2}} \qquad ( 2 )$$

where * represents complex conjugation.

Alternatively, the correlation coefficient may be obtained using another equalization of another type, such as those of the MMSE or MRC equalization, MMSE and MRC standing for Minimum Mean Square Error and Maximum Ratio Combining, respectively.

The correlation coefficient depends at least on frequency, on at least one spatial coordinate and on at least one electromagnetic property, and is representative of a correlation between the measured transfer function Hₘₙ(f) and the model Wₘₙ(f, rₚ, ε′ᵣ).

Steps 101, 102, 103 are iterated for each pair associating one transmit antenna of index m and one receive antenna of index n.

In step 104, an overall estimation function estimating the correlation coefficient is then determined for all the observations made by the radio-frequency detection system.

Given that the transfer functions are observed over a set of L discrete frequencies fₗ, and for the various bistatic angles between M transmitter positions rₘ and N receiver positions rₙ, the overall estimation function is obtained by taking a coherent sum of the various observations, such as represented by Equation (3):

$$ℒ ( {r}_{p} , {ε}_{r}^{′} ) = \frac{1}{M N L} {Σ}_{m = 1}^{M} {Σ}_{n = 1}^{N} {Σ}_{l = I}^{L} {ℒ}_{m n} ( {f}_{l} , {r}_{p} , {ε}_{r}^{′} ) \qquad ( 3 )$$

In step 105, at least one maximum of the overall estimation function, or more precisely of its absolute value or of its squared norm when this function is complex, is then sought. This search is carried out in the multidimensional space consisting of the spatial variables and of the variables characterizing the electromagnetic properties forming the domain of definition of this function.

This approach is based on the principle that the values of the electromagnetic properties that best correspond to the physical reality of the actual, real region of ground in question will result in values of the correlation coefficient that are consistent between the various frequencies and various observation configurations, maximizing the modulus of the coherent sum of Equation (3).

This principle of maximization of the modulus of the coherent sum of the correlation coefficients for values of variables that correspond to an observed physical reality is also called “focusing”.

In step 106 of the method, the value of at least one electromagnetic property associated with a spatial position is deduced.

For example, in the case of the example of Equations (2) and (3), a position of value

$${r}_{p}^{max}$$

and a dielectric permittivity of value

$${ε}_{r}^{′ max}$$

associated with this position are obtained, these values being defined by Equation (4):

$$( {r}_{p}^{max} , {ε}_{r}^{′ max} ) = {argmax}_{{r}_{p} , {ε}_{r}^{′}} \vert ℒ ( {r}_{p} , {ε}_{r}^{′} ) \vert \qquad ( 4 )$$

where argmax is the mathematical function argument of the maximum, which delivers the values of the variables for which a function reaches its maximum.

Given the predefined model expressed by Equation (1), the vector

$${r}_{p}^{max}$$

teaches the position of the point scatterer delivering the largest radar cross section in the observed space and

$${ε}_{r}^{′ max}$$

teaches the most representative value of the real part of the average dielectric permittivity in the part of the medium traversed by the electromagnetic wave between the transmit antennas and the receive antennas via the position

$${r}_{p}^{max} .$$

One drawback of the method described in FIG. 1 resides in the complexity of its solution, which is related to the multidimensional search space. Specifically, when the space of the dimensions is in 3D, the total search space is of 4 dimensions as it includes the search for the real part of the dielectric permittivity. Without predefined constraints on the search intervals of these various variables, the algorithm illustrated in FIG. 1 is complex to implement.

To overcome this drawback, a method of optimization under constraints is provided, to limit the search spaces.

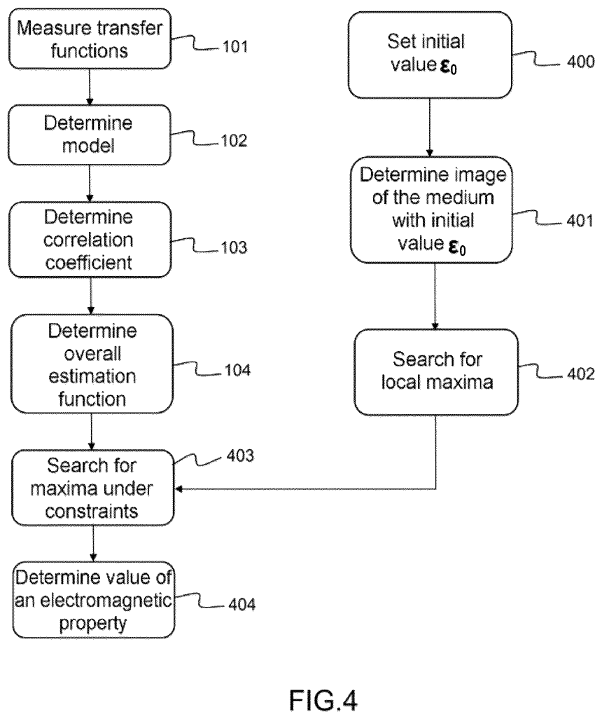

This method is described in FIG. 4 in the case of determination of the real part of the permittivity of a medium, a region of ground for example. The invention applies identically to other variables characterizing the medium, such as permeability.

Steps 101 to 104 are identical to those already described with reference to FIG. 1.

In parallel or successively, in step 400, a predefined initial value is set for the real part of the permittivity, denoted ε₀. For this value, a 2D or 3D image of the medium is determined (step 401) by means of a prior-art radar-imaging method such as the point-scatterer method. One example of such a method is described in the applicant's patent application FR3124607.

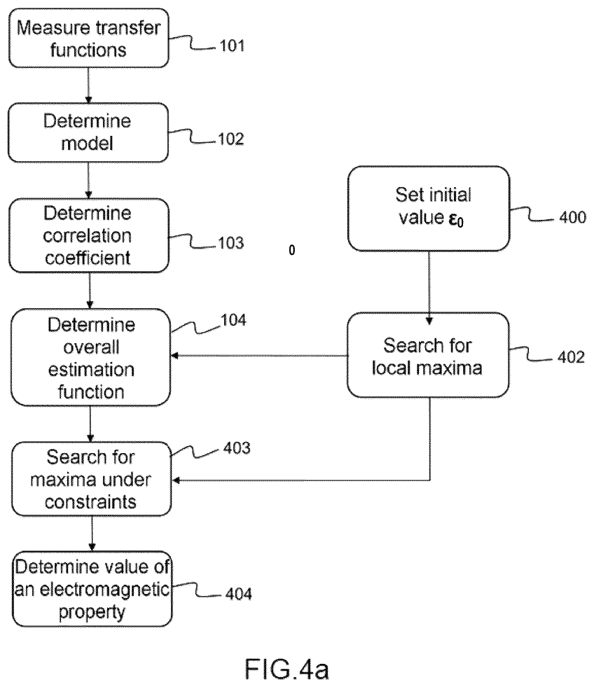

For example, if the target medium is a region of ground, the image is formed in 2D in the plane (x,z). One example of such an image is given in FIG. 5.

In step 402, the set of local maxima grouped in a set Ω={xᵢ, zᵢ}_{i=1 . . . P} are sought in the image, P being the number of local maxima.

For example, FIG. 5 shows one example of a local maximum 501 detected in step 402 for an initial permittivity ε₀=0.

In one variant of embodiment of the invention described in FIG. 4a, step 402 is performed not by searching for local maxima in a radar image but by searching for the local maxima directly in the overall estimation function determined in step 104, with the permittivity value set to its initial value in step 400. In this variant, the step 401 of determining an image of the medium is eliminated.

In step 403, the values of x,z and ε that maximize the overall estimation function determined in step 104 are then sought, with the following search intervals set:

$$\begin{matrix}( x , y , z ) = max {‖ ℒ ( x , z , ε ) ‖}^{2} \\ x ϵ [ {x}_{i} - {δ}_{x} ; {x}_{i} + {δ}_{x} ] \\ z ϵ [ {z}_{i} - {δ}_{z} ; {z}_{i} + {δ}_{z} ] \\ εϵ [ 1 ; {ε}_{max} ]\end{matrix}$$

δₓ, δ_{z} and εₘₐₓ defining search intervals.

Step 403 is executed for each local maximum of the set Ω.

In one particular embodiment, the overall estimation function is given by the relationship:

$$ℒ ( x , z , ε ) = \frac{1}{M N L} {Σ}_{m = 1}^{M} {Σ}_{n = 1}^{N} {Σ}_{l = 1}^{L} {ℒ}_{m n} ( {f}_{l} , x , z , ε ) {ℒ}_{m n} ( f , x , z , ε ) = \frac{{H}_{m n} ( f )}{ε f {d}_{m n}^{*} ( {x}_{𝔱} z )} {e}^{- i 2 π f \frac{\sqrt{ε}}{c} {d}_{m n} ( x , z )} {d}_{m n} ( x , z ) = \sqrt{{( x - {x}_{m} )}^{2} + {( z - {z}_{m} )}^{2}} + \sqrt{{( x - {x}_{n} )}^{2} + {( z - {z}_{n} )}^{2}} {d}_{m n}^{*} ( x , z ) = \sqrt{{( x - {x}_{m} )}^{2} + {( z - {z}_{m} )}^{2}} \times \sqrt{{( x - {x}_{n} )}^{2} + {( z - {z}_{n} )}^{2}} \qquad ( 5 )$$

where dₘₙ(x, z) is the sum of the respective distances between the point of coordinates (x,z) and the transmit antenna m and receive antenna n of the radar, respectively, and

$${d}_{m n}^{*}$$

(x, z) is the product of the respective distances between the point of coordinates (x,z) and the transmit antenna m and receive antenna n of the radar, respectively.

The method described in FIG. 4 has the advantage of limiting the search space since the search for maxima in the spatial-location space is limited to intervals in a neighbourhood around the positions detected in step 402 in the image of the medium.

However, it is still necessary to set the limits of the search intervals around these positions.

One variant of embodiment of the method consists in exploiting the concept of electrical length defined by the relationship: c(m, n)=\sqrt{ε}dₘₙ(x, z).

In theory, the electrical length associated with a local maximum of the image of the medium should remain constant regardless of the position x, z of the local maximum obtained with various predefined dielectric-permittivity values.

This property may be used to limit the size of the space in which the values of x and z are sought by ensuring they have the following property:

$${c}_{i} ( m , n ) = \sqrt{{ε}_{0}} {d}_{m n} ( {x}_{i} , {z}_{i} ) = \sqrt{ε} {d}_{m n} ( x , z ) \qquad ( 6 )$$

Equation (6) must be respected by all the local maxima (xᵢ, zᵢ) detected in step 402, for all the indices m,n of the respective positions of the transmit and receive antennas.

Thus, Equation (6) sets M×N constraints for each local maximum detected in step 402, M being the number of transmitters and N the number of receivers. These constraints make it possible to reduce the size of the space in which the three variables (x,z,ε) are sought. The optimization may also be reduced to a search for two variables since one of the three variables is related to the other two by Equation (6).

In another variant of embodiment of the invention, the impact of measurement noise on the value of the electrical length is taken into account. Specifically, the estimation of the electrical length cᵢ(m,n) may be noisy. To take this uncertainty into account, a constraint is added in the form of the following equations:

$$α {c}_{i} ( m , n ) - \sqrt{ε} {d}_{m n} ( x , z ) = 0 , ∀ n , ∀ m αϵ [ 1 - {δ}_{a} ; 1 + {δ}_{a} ] \qquad ( 7 )$$

where δ_{α} defines the interval of variation of the parameter α.

In yet another variant of embodiment, in order to further limit the search domain of the variables, Equation (6) is applied only to the pair of transmit and receive antennas that minimizes the distance dₘₙ(xᵢ, zᵢ). This allows the size of the space in which the variables are sought to be further decreased.

Although the above example related to the context of location in 2D space (x,z), the method is applicable in an identical manner to a 3D space (x,y,z). Constraints on the variable y are then added.

The search for maxima of the global estimation function is carried out by means of numerical optimization algorithms such as described for example in reference [11].

FIG. 5 shows one example of a result obtained by applying the method according to the invention.

A target was placed at (x=0.254; z=1.758) in a uniform medium of permittivity ε=4.255. Another target was placed in the medium to provide a disturbance. The second target was in position (1.207; 1.080). Its radiated power was four times lower than the first target's.

The first detection (step 402) was carried out with ε0=9. A local maximum in position (xᵢ₌₁=0.440; zᵢ₌₁=1.200) was identified (reference 501). Step 403 of the method was then applied and a maximum 502 in position (x=0.252; z=1.761; ε=4.242) was obtained, i.e. an error of less than one millimetre in respect of position and of the order of 0.01 in respect of permittivity.

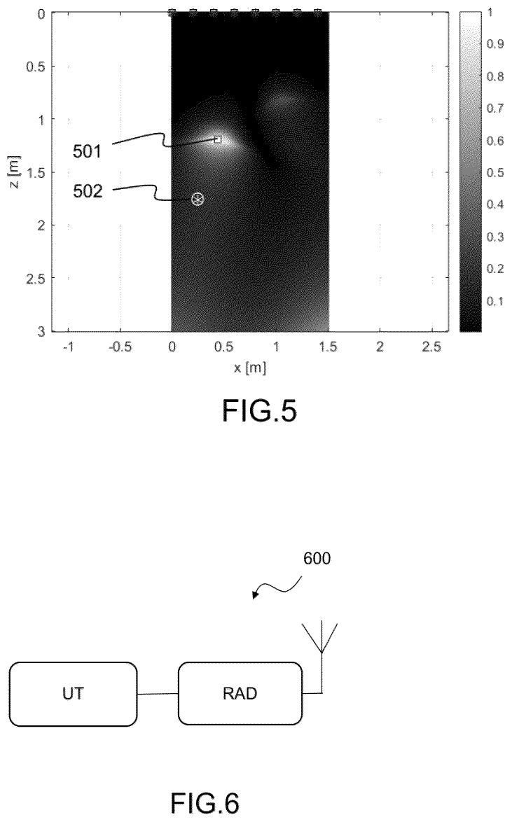

FIG. 6 schematically shows a system 600 for determining electromagnetic properties of a medium according to one embodiment of the invention.

The system 600 mainly comprises a radio-frequency detection device RAD of the type illustrated in FIG. 2 and a processing unit UT configured to implement the method according to the invention on the basis of measurements made by the device RAD.

The processing unit UT may take the form of software and/or hardware, and in particular employ one or more processors and one or more memories. The processor may be a generic processor, a specific processor, an application-specific integrated circuit (ASIC) or a field-programmable gate array (FPGA).

The system 600 may comprise a user interface for displaying results produced by the method.

### REFERENCES

[1]S. Serkan and V. Borecky, “Estimation methods for obtaining GPR signal velocity”, in Proceedings of the Third International Conference on ACSEE, Zurich, Switzerland, 2015, pp. 10-11.
[2]E. Forte and M. Pipan, “Review of multi-offset GPR applications: Data acquisition, processing and analysis”, Signal processing, vol. 132, pp. 210-220, 2017.
[3]I. Dal Bo, A. Klotzsche et al., “Geophysical imaging of regolith in landscapes along a climate and vegetation gradient in the Chilean coastal cordillera”, Catena, vol. 180, pp. 146-159, 2019.
[4]Q. Dou, L. Wei, D. R. Magee, and A. G. Cohn, “Real-time hyperbola recognition and fitting in GPR data”, IEEE Transactions on Geoscience and Remote Sensing, vol. 55, no. 1, pp. 51-62, 2017.
[5]C. Ozdemir, S, Demirci et al., “A review on migration methods in B-scan ground penetrating radar imaging”, Mathematical Problems in Engineering, vol. 2014, 2014.
[6]Z. Dong, X. Feng et al., “3D migration depth focus velocity analysis of hand-held ground penetrating radar”, Geosciences, vol. 12, no. 4, p. 178, 2022.
[7]E. F. Knott, J. F. Schaeffer, and M. T. Tulley, Radar Cross Section. SciTech Publishing, 2004.
[8] Demery, D. A., J. D. Parsons, and A. M. D. Turkmani. “Sounding techniques for wideband mobile radio channels: a review.” IEEE Proceedings (Communications, Speech and Vision) 138.5 (1991): 437-446.
[9] Laurenson, Dave, and Peter Grant. “A review of radio channel sounding techniques.” 2006 14th European Signal Processing Conference. IEEE, 2006.
[10] Ullah, M. Habib, and A. Unggul Priantoro. “A review on multiplexing schemes for MIMO channel sounding.” International Journal of Computer Science and Network Security 9.6 (2009): 294-300.
[11] Michael Grant and Stephen Boyd. CVX: Matlab software for disciplined convex programming, version 2.0 beta. https://cvxr.com/cvx, September 2013

1. A computer-implemented method for determining electromagnetic properties of a medium, comprising the steps of:
determining, by means of a radio-frequency detection system comprising at least one transmitter and one receiver, for each pair associating one receiver and one transmitter, a measurement of a frequency transfer function of a transmission channel characterizing said medium,
determining a model of said frequency transfer function dependent on at least one positional variable and on at least one variable characterizing the at least one electromagnetic property of the medium,
determining an estimation function estimating a correlation coefficient between the at least one measurement of a frequency transfer function and the model of said function,
setting an initial value of the at least one electromagnetic property,
searching for a set of first local maxima of the estimation function in the domain defined by the at least one positional variable for the initial value of the at least one electromagnetic property,
for each first local maximum, searching for a second local maximum of the estimation function in the domain defined by the at least one variable characterizing the at least one electromagnetic property of the medium and in a domain defined by the at least one positional variable constrained by the first local maximum.

2. The method for determining electromagnetic properties of a medium according to claim 1, wherein the search for a second local maximum of the estimation function is carried out over an interval of the at least one positional variable in the vicinity of the first local maximum.

3. The method for determining electromagnetic properties of a medium according to claim 1, wherein the search for a second local maximum of the estimation function is carried out in a constrained domain such that the electrical length, defined by the product of the square root of the real part of the dielectric permittivity and the total distance travelled by the signal between a transmitter and a receiver, remains substantially constant between the first local maxima and the second local maximum.

4. The method for determining electromagnetic properties of a medium according to claim 3, wherein the constraint of substantially constant electrical length is imposed for every pair associating one transmitter and one receiver.

5. The method for determining electromagnetic properties of a medium according to claim 3, wherein the constraint of substantially constant electrical length is imposed only for the pair associating one transmitter and one receiver that minimizes the distance travelled by a radio-frequency signal with a scatterer of coordinates equal to the first local maximum.

6. The method for determining electromagnetic properties of a medium according to claim 1, further comprising a step of determining an image of the medium as a function of the at least one positional variable from said measurements and the at least one initial value, the search for said set of first local maxima being carried out on the image and not the estimation function.

7. The method for determining electromagnetic properties of a medium according to claim 1, wherein the steps of determining at least one measurement of the frequency transfer function and of determining a model of said transfer function are carried out for each pair associating one transmitter and one receiver of the detection system, the method further comprising determining a correlation coefficient between said measurement and said model for each of said pairs, the estimation function being calculated from all the correlation coefficients.

8. The method for determining electromagnetic properties of a medium according to claim 7, wherein the amplitude and phase of the model of the frequency transfer function depend on the distance between a transmit antenna and a point on the plane and the distance between the point on the plane and a receive antenna.

9. The method for determining electromagnetic properties of a medium according to claim 1, wherein the at least one variable characterizing an electromagnetic property of the medium is selected from: the real part or imaginary part of the dielectric permittivity or permeability.

10. The method for determining electromagnetic properties of a medium according to claim 1, wherein the correlation coefficient is determined using a ZF, MMSE or MRC equalization method.

11. A device for determining electromagnetic properties of a medium comprising a radio-frequency detection system (RAD) comprising at least one transmitter and one receiver and a processing unit (UT), the system being configured to implement the method according to claim 1.

12. The device according to claim 11, wherein the radio-frequency detection system is a ground-penetrating radar.

13. A computer program comprising code instructions that cause a device to execute the steps of the method according to claim 1, the device for determining electromagnetic properties of a medium comprising a radio-frequency detection system (RAD) comprising at least one transmitter and one receiver and a processing unit (UT), wherein the system is configured to implement the method according to claim 1.

14. A computer-readable medium on which the computer program according to claim 13 is stored.
